(19) United States
(12) Patent Application Publication
(10) Pub. No.: US 2025/0171817 A1
(43) Pub. Date: May 29, 2025

(54) ENZYME COMBINATION, GENETICALLY ENGINEERED BACTERIA, AND APPLICATION THEREOF IN PRODUCING D-PSICOSE

(71) Applicant: HENAN ZHONGDA HENGYUAN BIOTECHNOLOGY STOCK CO., LTD., Luohe (CN)

(72) Inventors: Sanying WANG, Luohe (CN); Ziheng JIn, Luohe (CN); Yanjun Wen, Luohe (CN); Linzheng Li, Luohe (CN); Chenhui Wen, Luohe (CN); Rongrong Xie, Luohe (CN); Tianyi Pan, Luohe (CN)

(21) Appl. No.: 19/041,945

(22) Filed: Jan. 30, 2025

Related U.S. Application Data
(63) Continuation of application No. PCT/CN2024/108980, filed on Jul. 31, 2024.

(30) Foreign Application Priority Data
Jul. 19, 2023 (CN) 202310885692.1

(51) Int. Cl. C12P 19/02; C12N 9/90; C12N 9/92; C12P 19/24

(52) U.S. Cl. CPC C12P 19/02; C12N 9/90; C12N 9/92; C12P 19/24; C12Y 501/03; C12Y 503/01009

(57) ABSTRACT

The present invention relates to an enzyme combination, genetically engineered bacteria, and application thereof in producing D-psicose. In the present invention, glucose isomerase and D-psicose 3-epimerase from specific sources are co-expressed in Bacillus subtilis. A crude enzyme preparation is obtained by fermenting obtained engineered bacteria, and isomerization is performed by using high-concentration glucose as a substrate. Fructose syrup and D-psicose syrup are obtained by performing isolation, purification, and concentration. The enzyme combination provided in the present invention has a high catalytic rate for a substrate, and can clearly increase a conversion rate of D-psicose. In addition, inexpensive glucose is used as a raw material, so that the production costs of D-psicose are greatly reduced. In addition, fructose syrup is synchronously produced while D-psicose is produced, so that the input for a whole set of production lines of fructose syrup is omitted, thereby greatly improving the economic benefits.

This application is a Continuation Application of PCT/CN2024/108980, filed on Jul. 31, 2024, which claims priority to Chinese Patent Application No. 202310885692.1, filed on Jul. 19, 2023, which is incorporated by reference for all purposes as if fully set forth herein.

A Sequence Listing XML file named “10025_0123.xml” created on Jan. 30, 2025 and having a size of 26,851 bytes, is filed concurrently with the specification. The sequence listing contained in the XML file is part of the specification and is herein incorporated by reference in its entirety.

### TECHNICAL FIELD

The present invention relates to the field of bioengineering technologies, and in particular, to an enzyme combination, genetically engineered bacteria, and application thereof in producing D-psicose.

### BACKGROUND

D-psicose is a rare sugar having 70% sweetness of sucrose but low energy, and is an ideal substitute for sucrose. In addition, D-psicose has a lower absorption rate than other sweeteners, so that the absorption of fructose and glucose by an organism can be reduced, and the deposition of fat is reduced, thereby reducing the risks of type 2 diabetes, obesity, and other diseases. At present, there are also researches showing that D-psicose also has the functions of lowering blood lipid and blood sugar. The United States Food and Drug Administration (FDA) has approved D-psicose as generally recognized as safe (GRAS) and can be used as a component of a food additive. D-psicose has broad application prospect in food and health care products.

At present, fructose is mainly used as a raw material for psicose, and psicose is generated through enzyme catalysis of D-psicose 3-epimerase. However, the high market prices of fructose inevitably lead to high production costs of psicose. Fructose may be generated through epimerization of glucose catalyzed by glucose isomerase. The fermentation and application of the double-gene co-expressing engineered bacteria by using glucose isomerase and D-psicose 3-epimerase can implement the one-step generation of D-psicose from glucose. This method uses glucose as a raw material, thereby reducing raw material costs; can further omit one production line for producing fructose from glucose, thereby reducing equipment investment; and has important industrial application value.

### SUMMARY

In view of this, the present invention provides an enzyme combination, genetically engineered bacteria, and application thereof in producing D-psicose. In the present invention, glucose isomerase and D-psicose 3-epimerase from specific sources are used for catalyzing the conversion of a glucose substrate into D-psicose, which clearly improves the conversion efficiency of D-psicose, reduces the production time, and is suitable for industrial production.

To achieve the foregoing inventive objective, the following technical solutions are provided in the present invention:
The present invention provides an enzyme combination, including glucose isomerase derived from Thermus thermophilus and D-psicose 3-epimerase derived from Ruminococcus sp.

An amino acid sequence of the glucose isomerase is any one selected from the following:
(1) the sequence shown in SEQ ID NO: 1;
(2) an amino acid sequence that is obtained by substituting, deleting or adding one or more amino acids to/from the sequence shown in SEQ ID NO: 1 and has a function remaining unchanged; or
(3) an amino acid sequence that is at least 90% homologous with the sequence shown in SEQ ID NO: 1 and has protein activity same as or similar to that of the sequence shown in SEQ ID NO: 1.

An amino acid sequence of the D-psicose 3-epimerase is any one selected from the following:
(a) the sequence shown in SEQ ID NO: 2;
(b) an amino acid sequence that is obtained by substituting, deleting or adding one or more amino acids to/from the sequence shown in SEQ ID NO: 2 and has protein activity remaining unchanged; or
(c) an amino acid sequence that is at least 90% homologous with the sequence shown in SEQ ID NO: 2 and has protein activity same as or similar to that of the sequence shown in SEQ ID NO: 2.

In specific embodiments, the glucose isomerase GI in the present invention is derived from Thermus thermophilus, and has the registry number being WP_244348257.1 and the amino acid sequence being SEQ ID NO: 1. The D-psicose 3-epimerase DPE is derived from Ruminococcus sp., and has the registry number being MBS6425357.1 and the amino acid sequence being SEQ ID NO: 2.

In the present invention, through long-term research, it is found that rates of conversion of glucose into psicose by catalyzing a glucose substrate by using enzyme combinations from different sources are different, to eventually obtain an optimal combination with a clearly higher conversion rate than other combinations, namely, a combination of glucose isomerase derived from Thermus thermophilus and D-psicose 3-epimerase derived from Ruminococcus sp. Specifically, the amino acid sequence of the glucose isomerase is SEQ ID NO: 1, and the amino acid sequence of the D-psicose 3-epimerase is SEQ ID NO: 2.

The present invention further provides a nucleic acid combination encoding the enzyme combination, including a gene of GI and a gene of DPE. In the present invention, the gene of GI and the gene of DPE are optimized according to the codon preference of Bacillus subtilis. The sequence of the optimized gene of GI is SEQ ID NO: 3, and the sequence of the optimized gene of DPE is SEQ ID NO: 4.

The present invention further provides a gene expression cassette, including a promoter, a gene in the nucleic acid combination, and a terminator. The gene of GI and the gene of DPE in the nucleic acid combination are separately initiated by a constitutive promoter. In some embodiments, the constitutive promoter is a p43 promoter.

The present invention further provides an expression vector, including a backbone vector and the nucleic acid combination. Genes encoding glucose isomerase and D-psicose 3 -epimerase are located in the same backbone vector. Each gene is initiated by a promoter. The promoters may be the same (for example, the p43 promoter) or may be different.

In the expression vector in the present invention, the backbone vector is pWB980, pP43NMK, pYH-P43, or the like, or may include, but not limited to, a common vector of any other type in the field.

The present invention further provides engineered bacteria transfected or transformed with the expression vector.

In the present invention, original bacteria of the engineered bacteria are Bacillus subtilis, and are Bacillus subtilis WB600 in a specific embodiment.

The present invention further provides a method for constructing the engineered bacteria. The gene of the glucose isomerase and the gene of the D-psicose 3-epimerase are connected to the backbone vector to obtain a recombinant vector; and the recombinant vector is transferred into the Bacillus subtilis to obtain the engineered bacteria. The sequence of the gene of the glucose isomerase is shown in SEQ ID NO: 3, and the sequence of the gene of the D-psicose 3-epimerase is shown in SEQ ID NO: 4.

In some specific embodiments, the method for constructing the engineered bacteria is as follows:
(1) A protein sequence of glucose isomerase (GI, SEQ ID NO: 1) derived from Thermus thermophilus and a protein sequence of the D-psicose 3-epimerase (DPE, SEQ ID NO: 2) derived from Ruminococcus sp. are selected, and codon optimization is performed on encoded genes thereof, where the sequence of the optimized gene of GI is SEQ ID NO: 3, the sequence of the optimized gene of DPE is SEQ ID NO: 4, and whole gene synthesis is performed by GenScript Biotech. The two genes are connected to the vector pWB980 through homologous recombination to obtain recombinant plasmids pWB980-GI and pWB980-DPE.
(2) The plasmid pWB980-DPE is then used as a template for PCR amplification to obtain a p43-RBS-DPE segment, and the segment is connected to the vector pWB980-GI through homologous recombination to further obtain a recombinant plasmid pWB980-GI-DPE. The genes of GI and DPE are separately controlled to be constitutive intracellular expression through the p43 promoter.
(3) The recombinant plasmid pWB980-GI-DPE is transformed into the Bacillus subtilis WB600 to obtain recombined Bacillus subtilis WB600/GI-DPE.

Experiments show that the strain may be directly fermented for catalyzing glucose to produce fructose syrup and D-psicose, has a high catalytic rate for glucose, clearly shortens a reaction time, is conducive to the industrial production of psicose, and has high economic benefits.

The present invention further provides application of an enzyme combination, the nucleic acid combination, the expression vector or the engineered bacteria in producing D-psicose, or application of same in preparing blood sugar and lipid lowering products.

The present invention further provides a preparation method of psicose, where psicose is generated by catalyzing the reaction of a glucose substrate by using a fermented culture, an extract, or an extracted or isolated enzyme preparation of the engineered bacteria co-expressing glucose isomerase and D-psicose 3-epimerase in the present invention as a catalyst. The method specifically includes the following steps:
fermenting and culturing the engineered bacteria, and performing bacterial cell disruption to obtain a crude enzyme preparation containing the glucose isomerase and the D-psicose 3-epimerase;
adding the glucose substrate to the crude enzyme preparation to obtain invert syrup; and
sequentially filtering, purifying, chromatographically separating, and concentrating the invert syrup to separately obtain the D-psicose and fructose. In specific embodiments, the preparation method of D-psicose includes the following steps:
(1) inoculating the recombinant strain into an LB seed culture medium, and performing overnight cultivation at 37° C. and 200 rpm for enrichment, to obtain a seed broth with an OD600 value ranging from 3 to 6;
(2) inoculating the seed broth of the recombinant strain obtained in Step (1) into a fermentation medium (10 g of peptone, 5 g of yeast extract, 2.5 g of monopotassium phosphate, 15 g of potassium hydrogen phosphate, 0.1 g of manganese chloride tetrahydrate, 0.1 g of magnesium sulfate heptahydrate, and 6 g of glucose, and water is added to fix the volume to 1 L) according to a proportion of 0.1% (v/v) for fermentation, where a fermentation temperature is 37° C., and performing cultivation at 200 rpm for 48 h to obtain a fermentation broth;
(3) adding lysozyme to the fermentation broth obtained in Step (2) according to 0.1% 0 (w/v), and performing reactions at 37° C. and 200 rpm for 1 h for bacterial cell disruption to release endoenzyme to obtain a crude enzyme preparation;
(4) adding glucose according to a final concentration of 600 g/L to the crude enzyme preparation obtained in Step (3), and performing reactions at 60° C. for 24 h to obtain invert syrup;
(5) performing filtration and impurity removal by using a ceramic membrane with a pore size ranging from 8 nm to 10 nm on the invert syrup obtained in Step (4) with a flux of 50 L/m2/h to obtain a filtrate;
(6) performing filtration, impurity removal, and decoloration by using a nanofiltration membrane with a molecular weight cut-off ranging from 100 daltons to 300 daltons on the filtrate obtained in Step (5) with a flux of 10 L/m²/h to obtain a filtrate;
(7) passing the filtrate obtained in Step (6) through cation resin and anion resin sequentially to remove anions and cations to obtain a desalted sugar solution, where the model of the cation resin is D001-FD, and the model of the anion resin is D354-FD;
(8) chromatographically separating the desalted sugar solution in Step (7) to obtain an AD solution being a psicose sugar solution and a BD solution being a mixed sugar solution of glucose and fructose, where it is controlled in the process of the chromatographic separation that the concentration of the sugar solution ranges from 15% to 25%, the temperature ranges from 50° C. to 60° C., the pressure ranges from 0.2 MPa to 0.3 MPa, the water-material mass ratio is 1:2, and the throughput is 2.2 kg/kg/d; and
(9) concentrating the AD solution and the BD solution obtained in Step (8) to a solid content of 75% to obtain D-psicose syrup and fructose syrup.

Compared with the prior art, the present invention has the following beneficial effects:
(1) In the present invention, glucose is converted into D-psicose within a short time by using an enzyme combination from a specific source, which clearly reduces the production time, greatly improves the economic benefits, and is conducive to industrial production.
(2) In the present invention, inexpensive raw material glucose is used as a substrate, so that the production costs of D-psicose are greatly reduced.
(3) In the present invention, fructose syrup may be synchronously produced while D-psicose is produced, so that the input for a whole set of production lines of fructose syrup can be omitted.

### BRIEF DESCRIPTION OF THE DRAWINGS

FIG. 1 is a map of a recombinant plasmid pWB980-GI-DPE;

FIG. 2 is an electrophoresis image of SDS-PAGE, where tracks 1 and 2 respectively represent: 1: WB600/pWB980, and 2: WB600/pWB980-GI-DPE; and

FIG. 3 is a liquid detection chromatograph.

### DESCRIPTION OF THE EMBODIMENTS

The present invention provides an enzyme combination, genetically engineered bacteria, and application thereof in producing D-psicose. A person skilled in the art may refer to the content herein to appropriately improve process parameters for implementation. It is to be particularly noted that all similar replacements and modifications will be apparent to a person skilled in the art, and are all considered to be included in the present invention. The methods and applications of the present invention have been described by means of preferred embodiments, and it is obvious that a related person can implement and apply the technology of the present invention by making modifications or appropriate changes and combinations to the methods and applications herein without departing from the content, spirit, and scope of the present invention.

Experimental materials used in the present invention are all common commercially available products and can be purchased in the market.

The present invention is further described below with reference to the accompanying drawings.

### Embodiment 1: The Construction and Functional Verification of Engineered Bacteria Co-Expressing Glucose Isomerase (GI) and D-Psicose 3-Epimerase (DPE) in the Present Invention

A protein sequence of glucose isomerase (GI, SEQ ID NO: 1) derived from Thermus thermophilus and a protein sequence of D-psicose 3-epimerase (DPE, SEQ ID NO: 2) derived from Ruminococcus sp. are selected, where after codon optimization is performed on encoded genes, the sequence of the optimized gene of GI is SEQ ID NO: 3, the sequence of the optimized gene of DPE is SEQ ID NO: 4, and whole gene synthesis is performed by GenScript Biotech. The synthesized genes are separately connected to a pUC57 vector, and are respectively named pUC57-GI and pUC57-DPE.

PCR amplification is performed by using the synthesized recombinant plasmid pUC57-GI as a template and GI-F and GI-R as primers, and gel extraction and purification are performed to obtain a GI segment. PCR amplification is performed by using the synthesized recombinant plasmid pUC57-DPE as a template and DPE-F and DPE-R as primers, and gel extraction and purification are performed to obtain a DPE segment.

PCR amplification is performed by using the plasmid vector pWB980 as a template and pWB980-F1 and pWB980-R1 as primers, and gel extraction and purification are performed to obtain a linear plasmid gene segment P1. PCR amplification is performed by using the plasmid vector pWB980 as a template and pWB980-F2 and pWB980-R2 as primers, and gel extraction and purification are performed to obtain a linear plasmid gene segment P2.

The GI segment and the linear gene segment P1 of pWB980 are connected according to the specification of the homologous recombination kit, and a connection product is electrotransferred to a competent cell of the Bacillus subtilis WB600 to obtain recombinant Bacillus subtilis named WB600/GI. The recombinant plasmid is named pWB980-GI. Verification is performed through the PCR of a transformant colony and sequencing and analysis.

TABLE 1
Primer sequences of the present invention

| Primer name | Primer sequence 5′-3′ |
|---|---|
| GI-F | ATAAAAAAGGAGACATGAACGATGTATGAACC |
| | AAAACCCGA (SEQ ID NO: 7) |
| GI-R | TCTTGGAATTGTGCTGAAGCTTTAACCACGCA |
| | CCCCTAACAGATA (SEQ ID NO: 8) |
| pWB980-F1 | AGCTTCAGCACAATTCCAAGA |
| | (SEQ ID NO: 9) |
| pWB980-R1 | CGTTCATGTCTCCTTTTTTATGTACTG (SEQ |
| | ID NO: 10) |
| DPE-F | ATAAAAAAGGAGACATGAACGATGAAATATGG |
| | TATTTATTACGCTTATTGGG (SEQ ID |
| | NO: 11) |
| DPE-R | TCTTGGAATTGTGCTGAAGCTTAGACTTTCAA |
| | ATACATGTTTTACAAAGTG (SEQ ID NO: 12) |
| pWB980-F2 | AGCTTCAGCACAATTCCAAGA (SEQ ID |
| | NO: 13) |
| pWB980-R2 | CGTTCATGTCTCCTTTTTTATGTACTG (SEQ |
| | ID NO: 14) |
| p43-RBS-DPE-F | CTAACTCATAACCGAGAGGT |
| | AGCATTATTGAGTGGATGATTATATTCC |
| | (SEQ ID NO: 15) |
| p43-RBS-DPE-R | ACCTTTCAGCAACTAAAATA |
| | TTAGACTTCAAATACATGTTTTACAAAGTG |
| | (SEQ ID NO: 16) |
| pWB980-GI-F | TATTTTAGTTGCTGAAAGGTGCG (SEQ ID |
| | NO: 17) |
| pWB980-GI-R | ACCTCTCGGTTATGAGTTAGTTC (SEQ ID NO: |
| | 18) |
| GI-JD-F | ACAGCCATTGAACATACGGT (SEQ ID |
| | NO: 19) |
| GI-JD-R | CCTTGGTAACCGCTAGACT (SEQ ID NO: 20) |
| DPE-JD-F | ACAGCCATTGAACATACGGT (SEQ ID |
| | NO: 21) |
| DPE-JD-R | TTCCATGCCCATCATAATATCATATT (SEQ |
| | ID NO: 22) |
| GI-DPE-JD-F | ATCTGATCCTGAAAGAGCGT (SEQ ID |
| | NO: 23) |
| GI-DPE-JD-R | GCCTGTTTCTGGGTGTTCG (SEQ ID NO: 24) |

The DPE segment and the linear gene segment P2 of pWB980 are connected according to the specification of the homologous recombination kit, and a connection product is electrotransferred to a competent cell of the Bacillus subtilis WB600 to obtain recombinant Bacillus subtilis named WB600/DPE. The recombinant plasmid is named pWB980-DPE. Verification is performed through the PCR of a transformant colony and sequencing and analysis.

PCR amplification is performed by using the recombinant plasmid pWB980-DPE as a template and p43-RBS-DPE-F and p43-RBS-DPE-R as primers, and gel extraction and purification are performed to obtain a p43-RBS-DPE segment. PCR amplification is then performed by using the recombinant plasmid pWB980-GI as a template and pWB980-GI-F and pWB980-GI-R as primers, and gel extraction and purification are performed to obtain a linear plasmid gene segment P3.

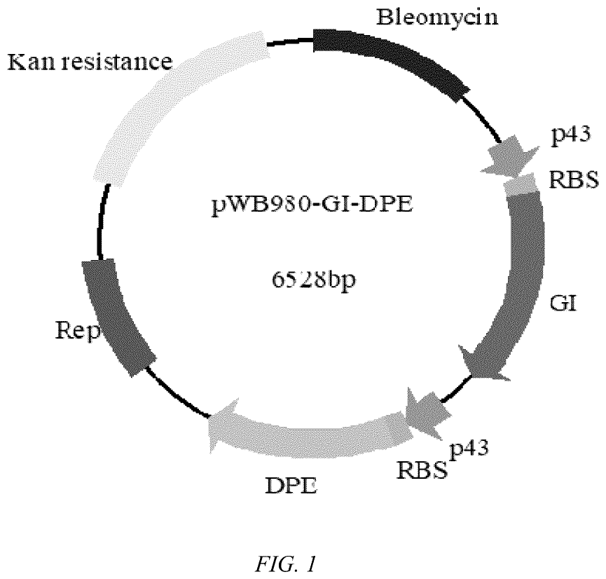

The p43-RBS-DPE segment and the linear gene segment P3 of pWB980-GI are connected according to the specification of the homologous recombination kit, and a connection product is electrotransferred to a competent cell of the Bacillus subtilis WB600 to obtain recombinant Bacillus subtilis named WB600/GI-DPE. The recombinant plasmid is named pWB980-GI-DPE (the map of the plasmid is shown in FIG. 1). Verification is performed through the PCR of a transformant colony and sequencing and analysis.

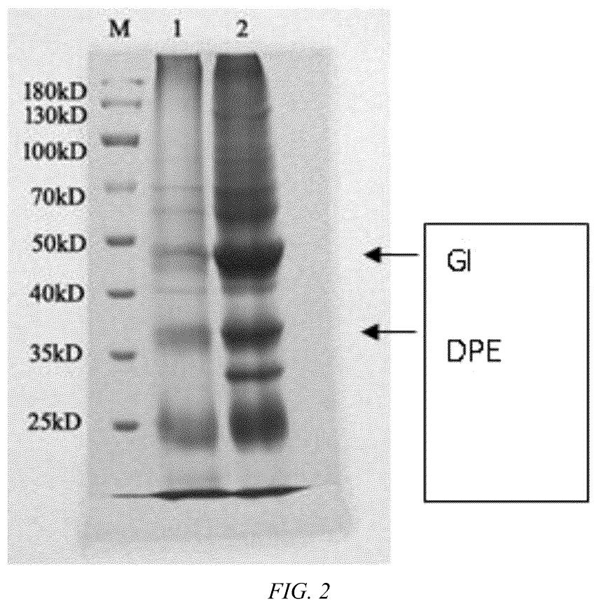

Positive transformants with correct PCR and sequencing of the colony are picked and inoculated into 5 ml of an LB liquid medium containing 50 mg/L of kanamycin for overnight cultivation at 37° C. and 200 rpm; and are inoculated into more than 1 L of a fermentation medium containing 50 mg/L of kanamycin with an inoculum size of 0.1% for cultivation at 37°° C. and 200 rpm for 48 h. Lysozyme powder is added according to 0.1% % to continue to perform reactions at 37° C. and 200 rpm for 1 h, and a GI-DPE crude enzyme preparation is obtained (an electrophoresis result of SDS-PAGE is shown in FIG. 2).

Glucose is added according to a final concentration of 700 g/L to the crude enzyme preparation obtained above for reactions at 60° C. for 24 h. 0.02 ml of a reaction solution is taken and diluted 50 times with pure water. Water bathing is performed at 100° C. for 10 min to inactivate enzyme. After centrifugation at 10000 rpm for 10 min, filtration is performed with a 0.22-μm microporous filter membrane. High performance liquid chromatography analysis is performed on the filtrate.

Bacillus subtilis WB600 with empty plasmids pWB980 is used for blank control, and other operation conditions are kept the same.

The high performance liquid chromatography is performed according to the following conditions: an Agilent high performance liquid chromatograph 1200; an analytical column: a Waters Sugar-Pak I chromatographic column; a mobile phase: pure water; a flow rate: 0.3 ml/min, and a column temperature: 80° C.; and a detector: a differential refractive index detector. The foregoing samples are analyzed by using pure substances of glucose, fructose, and D-psicose produced by the Sigma Corporation as standard substances, and a sample size is 10 μl.

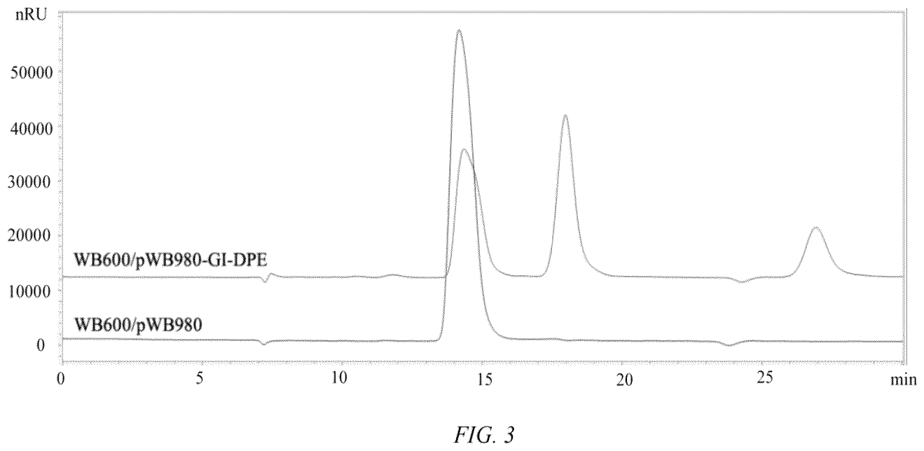

Liquid chromatography analysis results (FIG. 3) show that the peaking durations of glucose, fructose, and D-psicose are respectively 14.1 min, 17.9 min, and 26.9 min.

There is only glucose in the liquid chromatograph of the blank control (Bacillus subtilis WB600/pWB980); and there are glucose, fructose, and D-psicose in the liquid chromatograph of the experimental group (Bacillus subtilis WB600/pWB980-GI-DPE). The results show that the GI-DPE crude enzyme preparation prepared by using the foregoing method can convert glucose into fructose and then convert fructose into D-psicose.

Through the calculation of peak areas, after reactions for 24 h, the converted sugar solution contains 284 g/L of glucose, 209 g/L of fructose, and 107g/L of D-psicose, a ratio of which is 47.33:34.83:17.83.

### Embodiment 2: Method for Producing Fructose Syrup and D-Psicose Syrup Through Fermentation by Using Co-Expressing Engineered Bacteria in the Present Invention

Specific steps are as follows:
(1) inoculating the co-expressing engineered bacteria in the foregoing Embodiment 1 into an LB liquid seed culture medium containing 50 mg/L of kanamycin for overnight cultivation at 37° C. and 200 rpm to obtain a seed broth of the co-expressing engineered bacteria;
(2) inoculating the seed broth of the engineered bacteria obtained above into more than 1 L of a fermentation medium containing 50 mg/L of kanamycin (10 g of peptone, 5 g of yeast extract, 2.5 g of monopotassium phosphate, 15 g of potassium hydrogen phosphate, 0.1 g of manganese chloride tetrahydrate, 0.1 g of magnesium sulfate heptahydrate, and 6 g of glucose, and water is added to fix the volume to 1 L) according to a proportion of 0.1% (v/v) for fermentation, where a fermentation temperature is 37° C., and performing cultivation at 200 rpm for 48 h to obtain a fermentation broth;
(3) adding lysozyme to the fermentation broth obtained in Step (2) according to 0.1% 0 (w/v), and performing reactions at 37° C. and 200 rpm for 1 h for bacterial cell disruption to release endoenzyme to obtain a GI-DPE crude enzyme preparation.
(4) adding glucose according to a final concentration of 600 g/L to the crude enzyme preparation obtained in Step (3), and performing reactions at 60° C. for 2 h to 24 h to obtain invert syrup;
(5) performing filtration and impurity removal by using a ceramic membrane with a pore size ranging from 8 nm to 10 nm on the invert syrup obtained in Step (4) with a flux of 50 L/m2/h to obtain a filtrate;
(6) performing filtration, impurity removal, and decoloration by using a nanofiltration membrane with a molecular weight cut-off ranging from 100 daltons to 300 daltons on the filtrate obtained in Step (5) with a flux of 10 L/m²/h to obtain a filtrate;
(7) passing the filtrate obtained in Step (6) through cation resin and anion resin sequentially to remove anions and cations, where the model of the cation resin is D001-FD, and the model of the anion resin is D354-FD, to obtain a desalted sugar solution;
(8) chromatographically separating the desalted sugar solution in Step (7) to obtain an AD solution being a psicose sugar solution and a BD solution being a mixed sugar solution of glucose and fructose, where it is controlled in the process of the chromatographic separation that the concentration of the sugar solution ranges from 15% to 25%, the temperature ranges from 50°° C. to 60° C., the pressure ranges from 0.2 MPa to 0.3 MPa, the water-material mass ratio is 1:2, and the throughput is 2.2 kg/kg/d; and
(9) concentrating the AD solution and the BD solution obtained in Step (8) to a solid content of 75% to obtain D-psicose syrup and fructose syrup.

### Comparative Example 1

Glucose isomerase (GI, refer to SEQ ID NO: 1 in the records of CN113980880 for the nucleic acid sequence) and D-psicose 3-epimerase (DPE, refer to SEQ ID NO: 3 in the records of CN113980880 for the nucleic acid sequence) in Patent CN113980880 are selected. Bacillus subtilis co-expressing glucose isomerase and D-psicose 3-epimerase is constructed according to the method in Embodiment 1 of the present invention, and invert syrup containing glucose, fructose, and psicose is prepared according to the method in Embodiment 2 of the present invention.

### Comparative Example 2 (GI is Not Congeneric (is 56% Homologous With the GI in the Present Invention), and DPE is the Same)

Glucose isomerase (GI, refer to SEQ ID NO: 1 in the records of CN113980880 for the nucleic acid sequence) in Patent CN113980880 and D-psicose 3-epimerase (DPE, refer to SEQ ID NO: 4 for the nucleic acid sequence) derived from Ruminococcus sp. in this application are selected. Bacillus subtilis co-expressing GI and DPE is constructed according to the method in Embodiment 1 of the present invention. Invert syrup containing glucose, fructose, and psicose is prepared according to the method in Embodiment 2 of the present invention. Comparative example 3 (GI is not congeneric (97% homologous), and DPE is the same)

Glucose isomerase (GI, the registry number is WP_126200404.1, and the nucleic acid sequence is SEQ ID NO: 5 after codon optimization) derived from Thermus scotoductus and D-psicose 3-epimerase (DPE, the nucleic acid sequence is SEQ ID NO: 4) derived from Ruminococcus sp. in the present invention are selected. Bacillus subtilis co-expressing GI and DPE is constructed according to the method in Embodiment 1 of the present invention. Invert syrup containing glucose, fructose, and psicose is prepared according to the method in Embodiment 2 of the present invention.

### Comparative Example 4 (GI is the Same, and DPE is Different (is 40% Homologous With the DPE in the Present Invention))

Glucose isomerase (GI, the nucleic acid sequence is SEQ ID NO: 3) derived from Thermus thermophilus in the present invention and D-psicose 3-epimerase (DPE, refer to SEQ ID NO: 3 in the records of CN113980880 for the nucleic acid sequence) in Patent CN113980880 are selected. Bacillus subtilis co-expressing GI and DPE is constructed according to the method in Embodiment 1 of the present invention. Invert syrup containing glucose, fructose, and psicose is prepared according to the method in Embodiment 2 of the present invention.

### Comparative Example 5 (GI is the Same, and DPE is Different (is 69% Homologous With the DPE in the Present Invention))

Glucose isomerase (GI, the nucleic acid sequence is SEQ ID NO: 3) derived from Thermus thermophilus in the present invention and D-psicose 3-epimerase (DPE, WP 183684385.1, the nucleic acid sequence is SEQ ID NO: 6) derived from Oribacterium sinus are selected. Bacillus subtilis co-expressing GI and DPE is constructed according to the method in Embodiment 1. Invert syrup containing glucose, fructose, and psicose is prepared according to the method in Embodiment 2 of the present invention.

### Experimental Example

Invert syrup is prepared according to the methods in Embodiment 2 and Comparative examples 1 to 5. Samples of reactions for 2 h, 4 h, 6 h, 12 h, and 24 h are respectively taken. HPLC is performed to detect the content of glucose, fructose, and psicose, and the production rates of psicose are calculated. See Table 2 for results.

TABLE 2

| | | | Equilibrium | |
|---|---|---|---|---|
| | | Generated | conversion | Production |
| | | amount | rate (%) of | rate |
| | Reaction | (g/L) of | glucose and | (g/L/h) of |
| Group | time (h) | psicose | psicose | psicose |
| Embodiment | 0 | 0 | 17.83 | 0 |
| 1 | 2 | 107 | | 53.5 |
| | 4 | 107 | | 26.75 |
| | 6 | 107 | | 17.83 |
| | 12 | 107 | | 8.92 |
| | 24 | 107 | | 4.46 |
| Comparative | 0 | 0 | 15.83 | 0 |
| example 1 | 2 | 43 | | 21.5 |
| | 4 | 67 | | 16.75 |
| | 6 | 95 | | 15.83 |
| | 12 | 95 | | 7.92 |
| | 24 | 95 | | 3.96 |
| Comparative | 0 | 0 | 17.50 | 0 |
| example 2 | 2 | 50 | | 25 |
| | 4 | 83 | | 20.75 |
| | 6 | 105 | | 17.5 |
| | 12 | 105 | | 8.75 |
| | 24 | 105 | | 4.375 |
| Comparative | 0 | 0 | 17.83 | 0 |
| example 3 | 2 | 28 | | 14 |
| | 4 | 64 | | 16 |
| | 6 | 87 | | 14.5 |
| | 12 | 107 | | 8.92 |
| | 24 | 107 | | 4.46 |
| Comparative | 0 | 0 | | 0 |
| example 4 | 2 | 50 | 16.00 | 25 |
| | 4 | 96 | | 24 |
| | 6 | 96 | | 16 |
| | 12 | 96 | | 8 |
| | 24 | 96 | | 4 |
| Comparative | 0 | 0 | 14.00 | 0 |
| example 5 | 2 | 21 | | 10.5 |
| | 4 | 46 | | 11.5 |
| | 6 | 65 | | 10.83 |
| | 12 | 84 | | 7 |
| | 24 | 84 | | 3.5 |

As can be learned from the foregoing results, although conversion rates of producing psicose by catalyzing glucose with an enzyme in production of engineered strains in Comparative examples 1 to 5 and the present invention are close, all ranging from 14% to 18%, the durations required to reach the equilibrium conversion rate are clearly different, being respectively 6 h, 6 h, 12 h, 4 h, 12 h, and 2 h. In this case, it may be calculated that maximum production rates of psicose are respectively 21.5 g/L/h, 25 g/L/h, 16 g/L/h, 25 g/L/h, 11.5 g/L/h, and 53.5 g/L/h. The equilibrium catalytic duration of the enzyme combination of the present invention is the shortest, and is clearly lower than those in the other comparative examples, and the enzyme combination of the present invention also has the largest production rate of psicose, and is clearly higher than those of the other comparative examples. Therefore, it may be determined that the combination of glucose isomerase derived from Thermus thermophilus and D-psicose 3-epimerase derived from Ruminococcus sp. provided in the present invention has the highest catalytic rate for glucose and the highest production rate of psicose in the conversion reaction, and is the optimal combination.

The foregoing descriptions are preferred implementation manners of the present invention. It should be noted that for a person of ordinary skill in the art, several improvements and modifications may further be made without departing from the principle of the present invention. These improvements and modifications should also be deemed as falling within the protection scope of the present invention.

What is claimed is:

1. An enzyme combination, comprising glucose isomerase derived from Thermus thermophilus and D-psicose 3-epimerase derived from Ruminococcus sp.

2. The enzyme combination according to claim 1, wherein an amino acid sequence of the glucose isomerase is any one selected from the following:
(1) the sequence shown in SEQ ID NO: 1;
(2) an amino acid sequence that is obtained by substituting, deleting or adding one or more amino acids to/from the sequence shown in SEQ ID NO: 1 and has protein activity remaining unchanged; or (3) an amino acid sequence that is at least 90% homologous with the sequence shown in SEQ ID NO: 1 and has protein activity same as or similar to that of the sequence shown in SEQ ID NO: 1.

3. The enzyme combination according to claim 1, wherein an amino acid sequence of the D-psicose 3-epimerase is any one selected from the following:
(a) the sequence shown in SEQ ID NO: 2;
(b) an amino acid sequence that is obtained by substituting, deleting or adding one or more amino acids to/from the sequence shown in SEQ ID NO: 2 and has protein activity remaining unchanged; or
(c) an amino acid sequence that is at least 90% homologous with the sequence shown in SEQ ID NO: 2 and has protein activity same as or similar to that of the sequence shown in SEQ ID NO: 2.

4. A nucleic acid combination encoding the enzyme combination according to claim 1.

5. The nucleic acid combination according to claim 4, wherein
a gene of the glucose isomerase is a gene obtained by optimizing the codon preference of Bacillus subtilis, wherein a nucleotide sequence of the gene is shown in SEQ ID NO: 3; and
a gene of the D-psicose 3-epimerase is a gene obtained by optimizing the codon preference of Bacillus subtilis, wherein a nucleotide sequence of the gene is shown in SEQ ID NO: 4.

6. An expression vector, comprising a backbone vector and the nucleic acid combination according to claim 4.

7. Engineered bacteria containing the expression vector according to claim 6.

8. The engineered bacteria according to claim 7, wherein original bacteria of the engineered bacteria are Bacillus subtilis.

9. A method for constructing the engineered bacteria according to claim 7, wherein the gene of the glucose isomerase and the gene of the D-psicose 3-epimerase are connected to the backbone vector to obtain a recombinant vector; and the recombinant vector is transferred into the Bacillus subtilis to obtain the engineered bacteria.

10. A method of preparing D-psicose, comprising: making D-psicose by catalyzing a reaction of a glucose substrate by using a fermented culture, an extract, or an extracted or isolated enzyme preparation of the engineered bacteria according to claim 7 as a catalyst.

12. The method according to claim 11, further comprising:
fermenting and culturing the engineered bacteria, and performing bacterial cell disruption to obtain a crude enzyme preparation containing the glucose isomerase and the D-psicose 3-epimerase;
adding the glucose substrate to the crude enzyme preparation to obtain invert syrup; and
sequentially filtering, purifying, chromatographically separating, and concentrating the invert syrup to separately obtain the D-psicose and fructose.


SEQUENCE LISTING (24 sequences)

SEQ ID NO: 1 - protein, 387 aa, synthetic construct
MYEPKPEHRFTFGLWTVGNLGRDPFGDAVRERLDPVYVVHKLAELGAYGVNLHDEDLIPRGTPPQERDQIVRRFKKALEETGLKVPMVTANLFSDPAFKDGAFTSPDPWVRAYALRKSLETMDLGAELGAEIYVVWPGREGAEVEATGKARKVWDWVREALNFMAAYAEDQGYGYRFALEPKPNEPRGDIYFATVGSMLAFIHTLDRPERFGLNPEFAHETMAGLNFVHAVAQVLDAGKLFHIDLNDQRMSRFDQDLRFGSENLKAAFFLVDLLESSGYQGPRHFDAHALRTEDEEGVWAFARGCMRTYLILKERAEAFREDPEVKELLAAYYQQDPAALALLGPYSREKAEALKRAELPLEAKRRRGYGLERLDQLAVEYLLGVRG

SEQ ID NO: 2 - protein, 291 aa, synthetic construct
MKYGIYYAYWEKEWNGDYKYYIDKISKLGFDILEISCGAFSDYYTKDQELIDIGKYAKEKGVTLTAGYGPHLNESLSSSEPNTQKQAISFWKETLRKLKLMDIHMVGGALYGYWPVDYSKPFDKKRDLENSIKNMKIISQYAEEYDIMMGMEVLNRFEGYMLNTCDEALAYVEEVGSSNVGVMLDTFHMNIEEDNIAAAIRKAGDRLCHFHIGEGNRKVPGKGMLPWNEIGQALRDINYQHAAVMEPFVMQGGTVGHDIKIWRDIIGNCSEVTLDMDAQSALHFVKHVFEV

SEQ ID NO: 3 - DNA, 1164 bp, synthetic construct
  source 1..1164: mol_type = other DNA
atgtatgaaccaaaacccgagcacaggtttacgtttggcctgtggaccgttggtaatctgggccgtgatccatttggcgacgcggtgcgtgagcgtctggacccggtttatgtcgtgcacaaactggccgaactgggcgcctacggcgtgaacttgcatgatgaggaccttatcccgcgtggaaccccgcctcaggagcgggatcagattgtgcgccgtttcaagaaagctttagaagagactggtctgaaggttccgatggttaccgcaaatctgttcagcgatccggcgttcaaagatggtgcgttcacctcgccggatccgtgggtacgtgcatatgccttgaggaagtccctggagacgatggatttaggcgcggagctgggtgcggagatctacgtggtgtggccgggtcgcgagggcgctgaggttgaagcgaccggtaaggctcgtaaggtgtgggactgggttcgtgaagcgctcaacttcatggctgcgtatgcagaggaccagggctacggttaccgctttgcgttggagccgaagccgaatgaaccgagaggcgatatttactttgccacggtgggctccatgctggcgttcatccacaccctggacagacctgaacgctttggtctgaacccggaatttgctcatgaaaccatggcaggtttgaactttgttcatgcggtggcgcaggttctggacgccggtaaactgtttcatattgatctcaacgatcaacgtatgagccgtttcgaccaagacctgcgtttcggcagcgaaaacctgaaggcagcattcttcctggttgatttgctggagtctagcggttaccaaggtccgcgtcattttgacgcgcacgcgctgcgcaccgaagacgaagagggtgtttgggcgtttgcacgtggctgcatgcgtacctatctgatcctgaaagagcgtgcagaggcgttccgcgaggacccggaagtcaaagaactgttggccgcgtattatcagcaggatccggcagcgttggcgttgctgggtccatacagccgtgaaaaagcggaggcacttaagcgcgcggaactgccgctggaagctaaacgccgtcgtggttacggcctggagcgcctggaccaactggccgtcgaatatctgttaggggtgcgtggttaa

SEQ ID NO: 4 - DNA, 876 bp, synthetic construct
  source 1..876: mol_type = other DNA
atgaaatatggtatttattacgcttattgggaaaaggaatggaatggagattacaaatattatatagataaaattagcaaactgggctttgatattctggaaattagctgcggcgcgtttagcgattattataccaaagatcaggaactgattgatattggcaaatatgcgaaagaaaaaggcgtgaccctgaccgcgggctatggcccgcatctgaacgaaagcctgagcagcagcgaaccgaacacccagaaacaggcgattagcttttggaaagaaaccctgcgcaaactgaaactgatggatattcatatggtgggcggcgcgctgtatggctattggccggtggattatagcaaaccgtttgataaaaaacgcgatctggaaaacagcattaaaaacatgaaaattattagccagtatgcggaagaatatgatattatgatgggcatggaagtgctgaaccgctttgaaggctatatgctgaacacctgcgatgaagcgctggcgtatgtggaagaagtgggcagcagcaacgtgggcgtgatgctggatacctttcatatgaacattgaagaagataacattgcggcggcgattcgcaaagcgggcgatcgcctgtgccattttcatattggcgaaggcaaccgcaaagtgccgggcaaaggcatgctgccgtggaacgaaattggccaggcgctgcgcgatattaactatcagcatgcggcggtgatggaaccgtttgtgatgcagggcggcaccgtgggccatgatattaaaatttggcgcgatattattggcaactgctctgaagttacattagatatggacgctcaaagtgcgttgcactttgtaaaacatgtatttgaagtctaa

SEQ ID NO: 5 - DNA, 1164 bp, synthetic construct
  source 1..1164: mol_type = other DNA
atgtatgaaccgaaaccggaacatcgctttacctttggcctgtggaccgtgggcaacgtgggccgcgatccgtttggcgatgcggtgcgcgaacgcctggatccggtgtatgtggtgcataaactggcggaactgggcgcgtatggcgtgaacctgcatgatgaagatctgattccgcgcggcaccccgccgcaggaacgcgatcagattgtgcgccgctttaaaaaagcgctggaagaaaccggcctgaaagtgccgatggtgaccgcgaacctgtttagcgatccggcgtttaaagatggcgcgtttaccagcccggatccgtgggtgcgcgcgtatgcgctgcgcaaaagcctggaaaccatggatctgggcgcggaactgggcgcggaaatttatgtggtgtggccgggccgcgaaggcgcggaagtggaagcgaccggcaaagcgcgcaaagtgtgggattgggtgcgcggcgcgctgaactttatggcggcgtatgcggaagatcagggctatggctatcgctttgcgctggaaccgaaaccgaacgaaccgcgcggcgatatttattttgcgaccgtgggcagcatgctggcgtttattcataccctggatcgcccggaacgctttggcctgaacccggaatttgcgcatgaaaccatggcgggcctgaactttgtgcatgcggtggcgcaggtgctggatgcgggcaaactgtttcatattgatctgaacgatcagaaaatgagccgctttgatcaggatctgcgctttggcagcgaaaacctgaaagcggcgttttttctggtggatctgctggaaagcagcggctatcagggcccgcgccattttgatgcgcatgcgctgcgcaccgaagatgaagaaggcgtgtgggcgtttgcgcgcggctgcatgcgcacctatctgattctgaaagaacgcgcgaaagcgtttcgcgaagatccggaagtgaaagaaattctggcgacctattatgaagaagatccggaagcgattggcctgctgggcccgtatagccgcgaaaaagcggaagcgctgaaacgcgcggaactgccgctggaagcgaaacgccgccgcggctatgatctggaacgcctggatcagctggcggtggaatatctgctgggcgtgcgcggctaa

SEQ ID NO: 6 - DNA, 876 bp, synthetic construct
  source 1..876: mol_type = other DNA
atgaaatatggcatttattatgcgtattgggaaaaagaatggaccggcgattattttaaatatattgataaagtgaaagaactgggctttgatattctggaaattagctgcggcgcgtttaaaaaagattataccaccgatgaacagctgctgcgcattggcgaatatgcgaaagaaaaaggcattattctgaccgcgggctatggcccgcagaaagatgaaaacctggcgagcccggatgaagcggtgcagaaaaacgcgattgaattttttaaagaaaccctgcgcaaactgcatctgatgaacattcatattctgggcggcggcctgtatagctattggccggtggattttaacagcgatattaacaaagaacgcgattttgaaaccagcgtgaaaaacatgcgcctgattgcgaaatatgcggaagaatatgatattaccctgggcatggaagtgctgaaccgctttgaaggctatctgattaacacctgcgatgaatgcctgcgctatattgatgcggtgggcaacaaccgcgtgggcgtgatgctggatacctttcatatgaacattgaagaagataacattggcgatgcgattcgcaaagcgggcgataaactggtgcattttcatatgggcgaacgcaaccgcaaagtgccgggcaaaggcaccatgccgtgggcggaaattggccaggcgctgcgcgatattcattatcagcatgcggcggtgatggaaccgtttgtgctgcagggcggcaaagtgggccaggatattaaaatttggcgcaacctgctgccggataccagcgaagcgaaactggatgcggatgcgaaacatagctgcgaatttgtgcgccatgtgtttgaactgtaa

SEQ ID NO: 7 - DNA, 41 bp, synthetic construct
  source 1..41: mol_type = other DNA
ataaaaaaggagacatgaacgatgtatgaaccaaaacccga

SEQ ID NO: 8 - DNA, 45 bp, synthetic construct
  source 1..45: mol_type = other DNA
tcttggaattgtgctgaagctttaaccacgcacccctaacagata

SEQ ID NO: 9 - DNA, 21 bp, synthetic construct
  source 1..21: mol_type = other DNA
agcttcagcacaattccaaga

SEQ ID NO: 10 - DNA, 27 bp, synthetic construct
  source 1..27: mol_type = other DNA
cgttcatgtctccttttttatgtactg

SEQ ID NO: 11 - DNA, 52 bp, synthetic construct
  source 1..52: mol_type = other DNA
ataaaaaaggagacatgaacgatgaaatatggtatttattacgcttattggg

SEQ ID NO: 12 - DNA, 51 bp, synthetic construct
  source 1..51: mol_type = other DNA
tcttggaattgtgctgaagcttagactttcaaatacatgttttacaaagtg

SEQ ID NO: 13 - DNA, 21 bp, synthetic construct
  source 1..21: mol_type = other DNA
agcttcagcacaattccaaga

SEQ ID NO: 14 - DNA, 27 bp, synthetic construct
  source 1..27: mol_type = other DNA
cgttcatgtctccttttttatgtactg

SEQ ID NO: 15 - DNA, 48 bp, synthetic construct
  source 1..48: mol_type = other DNA
ctaactcataaccgagaggtagcattattgagtggatgattatattcc

SEQ ID NO: 16 - DNA, 50 bp, synthetic construct
  source 1..50: mol_type = other DNA
acctttcagcaactaaaatattagacttcaaatacatgttttacaaagtg

SEQ ID NO: 17 - DNA, 23 bp, synthetic construct
  source 1..23: mol_type = other DNA
tattttagttgctgaaaggtgcg

SEQ ID NO: 18 - DNA, 23 bp, synthetic construct
  source 1..23: mol_type = other DNA
acctctcggttatgagttagttc

SEQ ID NO: 19 - DNA, 20 bp, synthetic construct
  source 1..20: mol_type = other DNA
acagccattgaacatacggt

SEQ ID NO: 20 - DNA, 19 bp, synthetic construct
  source 1..19: mol_type = other DNA
ccttggtaaccgctagact

SEQ ID NO: 21 - DNA, 20 bp, synthetic construct
  source 1..20: mol_type = other DNA
acagccattgaacatacggt

SEQ ID NO: 22 - DNA, 26 bp, synthetic construct
  source 1..26: mol_type = other DNA
ttccatgcccatcataatatcatatt

SEQ ID NO: 23 - DNA, 20 bp, synthetic construct
  source 1..20: mol_type = other DNA
atctgatcctgaaagagcgt

SEQ ID NO: 24 - DNA, 19 bp, synthetic construct
  source 1..19: mol_type = other DNA
gcctgtttctgggtgttcg